(19) United States
(12) Patent Application Publication
(10) Pub. No.: US 2024/0164386 A1
(43) Pub. Date: May 23, 2024

(54) METHOD OF CONTROLLING RIPTORTUS PEDESTRIS BY ENHANCHING BIOLOGICAL ACTIVITY OF BEAUVERIA BASSIANA ARP14 AND COMPOSITION FOR CONTROL THEREOF

(71) Applicant: Andong National University Industry-Academic Cooperation Foundation, Andong-si (KR)

(72) Inventors: Un Taek LIM, Andong-si (KR); Souvic Sarker, Andong-si (KR)

(73) Assignee: Andong National University Industry-Academic Cooperation Foundation, Andong-si (KR)

(21) Appl. No.: 18/485,305

(22) Filed: Oct. 11, 2023

(30) Foreign Application Priority Data
Nov. 23, 2022 (KR) 10-2022-0158505

(51) Int. Cl. A01N 63/30; A01N 43/22; A01P 7/04; C12N 1/14

(52) U.S. Cl. CPC A01N 63/30; A01N 43/22; A01P 7/04; C12N 1/145; C12R 2001/645

(57) ABSTRACT

Proposed are a method of controlling Riptortus pedestris by enhancing a biological activity of Beauveria bassiana ARP14 and a controlling composition thereof. The control method includes co-treating Riptortus pedestris with azadirachtin and the Beauveria bassiana ARP14 strain (KCTC 12979BP). The control significantly increases the infectivity and mortality effect of the Beauveria bassiana ARP14 strain on Riptortus pedestris by enhancing the bioactivity of the Beauveria bassiana ARP14 strain through co-treatment with the Beauveria bassiana ARP14 strain and azadirachtin, especially the early infectivity and early mortality rate. The control method and composition can be widely used as safe and effective control measures against Riptortus pedestris.

### STATEMENT REGARDING GOVERNMENT SPONSORED RESEARCH

This invention was supported by the Institute of Planning and Evaluation for Technology in Food, Agriculture, and Forestry (IPET) funded by the Ministry of Agriculture, Food, and Rural Affairs (MAFRA). [Research Program name: “Agricultural Machinery/Equipment Localization Technology Development Program”; Research Project name: “Development of Technology for Maximization and Evaluation of Activity of Beauveria Strain”; Project Serial Number: 1545027152; Research Project Number: 321054053HD020]

### CROSS REFERENCE TO RELATED APPLICATION

The present application claims priority to Korean Patent Application No. 10-2022-0158505, filed Nov. 23, 2022, the entire contents of which is incorporated herein for all purposes by this reference.

### BACKGROUND OF THE DISCLOSURE

### 1. Field of the Disclosure

The present disclosure relates to a method for controlling Riptortus pedestris by enhancing biological activity of Beauveria bassiana ARP14 and a controlling composition thereof.

### 2. Description of the Related Art

With the increase in pest resistance to synthetic pesticides, a growing consumer demand for safe agricultural produce, and increase in the importance of environmental protection through environment-friendly agriculture, there is a growing need to develop microbial pesticides utilizing entomopathogenic microorganisms. Entomopathogenic fungi are composed of hyphae, which take the form of single cells or branched filaments, and mycelia which are masses of thread-like hyphae. Entomopathogenic fungi infiltrate into host insects through the body wall. In other words, the conidia of the entomopathogenic fungus attach to the epidermis of the host insect, and the conidia germinate, causing the hyphae to penetrate through the body wall of the host insect and into the interior of the insect. After invading the body of the host insect, the hyphae develop in the host body fluid, form mycelium, and invade the tissue body, blocking the host insect's immune system and secreting toxic substances to kill the host insect.

Entomopathogenic fungi have selectivity to eliminate only target pests, so they are generally safe, posing no direct or indirect harm to humans, higher organisms, or plants. It also has the advantage of minimizing the number of repeated applications of mycoinsecticide, as the mortality effect of a secondary infection is sustained. To date, more than 700 species of entomopathogenic fungi have been identified, and some of these entomopathogenic fungi, such as Beauveria bassiana, Metarhizium anisopliae, and Nomuraea rileyi, have been developed as microbial pesticides to control numerous pests.

Beauveria bassiana is a fungus belonging to the genus Beauveria from the Beauveriaceae family, and Beauveria bassiana is one of the best known entomopathogenic fungi. Beauveria bassiana has activity against a diverse group of insects, including moths. Currently, the most notable and available insecticide using Beauveria bassiana is Botanigard (USA), which was developed from a strain of Beauveria bassiana GHA. Korean Patent No. 10-0239209 describes Beauveria bassiana CS-1 strain (KFCC-10988) as a strain with an excellent mortality activity against the cabbage codling moth, and Korean Patent No. 10-1161368 describes Beauveria bassiana KK5 strain as a strain with a mortality activity against aphids, especially peach aphid (Myzuspersicae). However, even Beauveria bassiana, one of the most known and used entomopathogenic fungi, has been limited in use due to its limited applicable types of target pests, provides poor mortality activity compared to chemical synthetic insecticides because of the fact that it is affected by environmental conditions, especially temperature and humidity.

On the other hand, Riptortus pedestris, is a pest from the Alydidae family, found in Korea, Japan, and Taiwan where it mainly harms Fabaceae. Although Riptortus pedestris did little damage in the past, it has recently become a major problem nationwide since climate warming increased the survival of overwintering populations, and the use of pesticides has reduced natural enemies. The Riptortus pedestris primarily sucks the sap out of the beans as it attacks the pods from Fabaceae, so if the pods are attacked in the early stage of formation, the plants of the pods are stunted, dried up, and deformed. The Riptortus pedestris is difficult to control because of its ability to move to nearby crop covers or other areas during chemical spraying and then come back when the spraying is finished.

It would be desirable to control Riptortus pedestris using entomopathogenic fungi that are relatively safe for humans and the environment while having the selectivity to kill only target pests. Accordingly, the inventor applied for a patent on Feb. 26, 2016, for a new strain of microorganism Beauveria bassiana ARP14, which is recovered from Riptortus pedestris and has a high control efficacy against Riptortus pedestris, and registered Patent No. 10-1804038 (hereinafter referred to as the ‘First-to-File Patent’). Although the Beauveria bassiana ARP14 strain shows a higher control efficacy against Riptortus pedestris than existing Beauveria bassiana strains, it has poorer mortality activity compared to chemical synthetic insecticides, especially in the early stage, which is a major disadvantage in control of Riptortus pedestris when an early control is important.

Azadirachtin is a compound belonging to the limonoid group and is a secondary metabolic product in neem seeds. Azadirachtin is a highly oxidized Tetranortriterpenoid with a complex molecular structure that includes Enol ethers, Acetals, Hemiacetals, Tetrasubstituted epoxides, and various Carboxylic acid esters. Azadirachtin is known to affect more than 200 species of insects primarily as an anti-feeding agent and a growth disruptor. Azadirachtin meets many criteria for a good insecticide, being biodegradable with a very low toxicity to mammals. Because of those benefits, Azadirachtin is used in combination with many insecticides. However, when it comes to the activity of entomopathogenic fungi, studies to date have shown synergistic effects (Jallow et al. 2019 J. Appl. Entomol. 143:187), antagonistic effects (Mohan et al. 2007 Biocon. Sci. Tech. 17:1509), and no effect (Aristizabal et al. 2017 J. Appl. Entomol. 141:274), making it difficult to generalize the effects of combinations with entomopathogenic fungi, and the mechanism of action is unclear.

### RELATED ART DOCUMENTS

### Patent Document

(Patent Document 0001) Korean Patent No. 10-0239209
(Patent Document 0002) Korea Patent No. 10-1161368
(Patent Document 0003) Korea Patent No. 10-1804038

### Non-Patent Document

(Non-patent Document 0001) Jallow et al. 2019 J. Appl. Entomol. 143:187
(Non-patent Document 0002) Mohan et al. 2007 Biocon. Sci. Tech. 17:1509
(Non-patent Document 0003) Aristizabal et al. 2017 J. Appl. Entomol. 141:274

### SUMMARY OF THE DISCLOSURE

The present disclosure aims to provide a control method that enhances a bioactivity of Beauveria bassiana ARP14 to increase an infectivity and mortality effect against Riptortus pedestris, and a controlling composition thereof.

In particular, the present disclosure aims to provide a control method that enhances the bioactivity of Beauveria bassiana ARP14 to significantly increase an early infectivity and mortality effect against Riptortus pedestris, and a controlling composition thereof.

To accomplish the objectives, the present disclosure provides a method of controlling Riptortus pedestris through co-treatment with the Beauveria bassiana ARP14 strain (KCTC 12979BP) and azadirachtin.

The control method increases the early infectivity of the Beauveria bassiana ARP14 strain to Riptortus pedestris through the co-treatment. The control method particularly preferably results in a maximum mycosis rate within 10 days of the co-treatment of Riptortus pedestris.

In the control method, the Beauveria bassiana ARP14 strain is preferably contained in an amount of 1×10⁷ to 1×10⁹ spores in a unit dose (mL).

In the control method, azadirachtin is preferably diluted at 0.03 g/L to 0.2 g/L when used in the treating.

In one preferred embodiment of the control method, a 0.005% to 0.05% Triton X-100 solution is used as a solvent for the Beauveria bassiana ARP14 strain and azadirachtin.

In addition, the present disclosure provides a composition for controlling Riptortus pedestris, the composition containing the Beauveria bassiana ARP14 strain (KCTC12979BP) and azadirachtin as active ingredients in order to increase the early mycosis rate of Riptortus pedestris.

The composition contains the Beauveria bassiana ARP14 strain in an amount of 1×10⁷ to 1×10⁹ spores in a unit dose (mL), and azadirachtin that is diluted at 0.03 g/L to 0.2 g/L.

The control method of the present disclosure against Riptortus pedestris can significantly increase the infectivity and mortality effect on Riptortus pedestris by enhancing the bioactivity of the Beauveria bassiana ARP14 strain through co-treatment with the Beauveria bassiana ARP14 strain and azadirachtin. In particular, the early infectivity and early mortality rate of the Beauveria bassiana ARP14 strain against Riptortus pedestris can be significantly increased. The control method and composition of the present disclosure can exhibit a high early mycosis rate and mortality effect against Riptortus pedestris with the use of only the azadirachtin, which is an eco-friendly natural insecticide, and an insect pathogenic fungus that is safe for humans and the environment and has selectivity to remove only the target pests. Therefore, the control method and control composition of the present disclosure are safe and effective control measures.

### BRIEF DESCRIPTION OF THE DRAWINGS

FIG. 1 shows a result of comparison in mortality effect of a co-treatment with azadirachtin and a Beauveria bassiana ARP14 strain versus each treatment with one of the components alone; and

FIG. 2 shows a result of comparison in changes in infection rate through a co-treatment of azadirachtin with the Beauveria bassiana ARP14 strain versus each treatment with one of the components alone.

### DESCRIPTION OF THE PREFERRED EMBODIMENTS

The present disclosure relates to a method of controlling Riptortus pedestris by enhancing a bioactivity of a Beauveria bassiana ARP14 strain and, in particular, to a method of enhancing an early infectivity and mortality effect against Riptortus pedestris by significantly enhancing the control against Riptortus pedestris. The present disclosure also relates to a controlling composition against Riptortus pedestris for the purpose. Herein below, the control method and the controlling composition each will be described in detail using preferred embodiments of the present disclosure.

A method of controlling Riptortus pedestris in the present disclosure includes co-treating Riptortus pedestris with the Beauveria bassiana ARP14 strain (KCTC 12979BP) and azadirachtin.

The Beauveria bassiana ARP14 strain is a microorganism described as a new strain in Korean Patent No. 10-1804038, which was filed by the present inventors and granted prior to the date of the present patent application. The strain has been deposited in the Korean Agricultural Culture Collection (KACC), Agricultural Microbiology Division, National Institute of Agricultural Sciences under accession number KCTC 12979BP on Feb. 18, 2016. The Beauveria bassiana ARP14 strain is isolated from Riptortus pedestris and shows a higher control effect against Riptortus pedestris than existing Beauveria bassiana strains. However, this strain has less insecticidal power, especially early insecticidal power, than other chemically synthesized insecticides.

Azadirachtin is a secondary metabolite present in neem seeds and is known to affect many insects by acting as an anti-feeding agent and growth disruptor. Azadirachtin is a component of an environmentally friendly natural insecticide which is biodegradable and exhibits a very low toxicity to mammals. In one embodiment of the present disclosure, Azatrol™ (Azatrol EC, PBI-GordonCorporation; with 1.2% of Azadirachtin contained) was used as an active ingredient.

The control method of the present disclosure enhances a mortality effect against Riptortus pedestris by co-treating Riptortus pedestris with the above-mentioned Beauveria bassiana ARP14 strain and azadirachtin. In particular, the co-treatment with azadirachtin may increase an early infection rate of the Beauveria bassiana ARP14 strain against Riptortus pedestris, which may significantly increase an early mortality rate of Riptortus pedestris. As a result, the control effect against Riptortus pedestris may be significantly enhanced. It is especially important to achieve a mortality effect early in the control process because, as in the case of Fabaceae, a failure to achieve a mortality effect early in the control leads to an early damage to the crop significantly and often leads to an irreversible damage. In addition, without a mortality effect at the beginning, it is difficult to control Riptortus pedestris because they move to nearby fields or other areas and then come back.

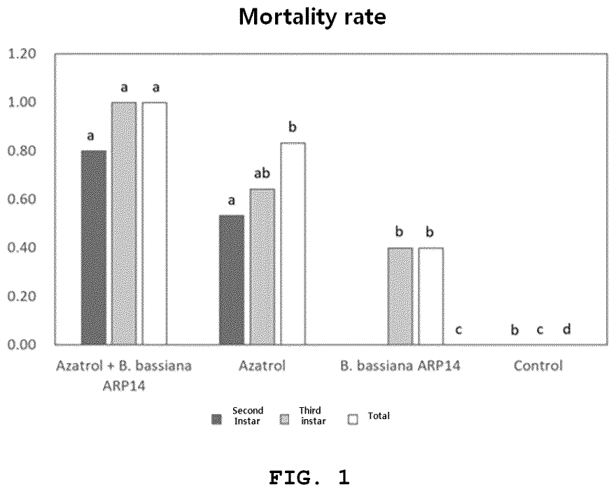

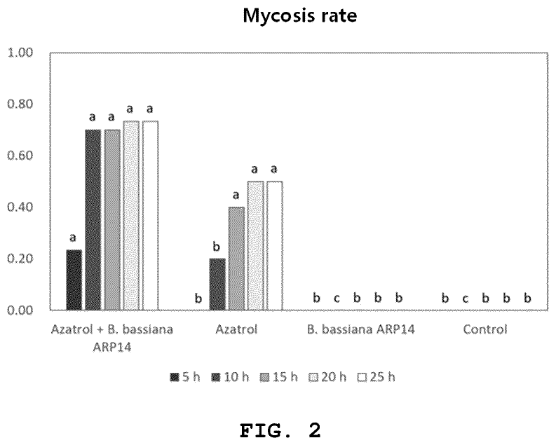

As confirmed in the experimental examples below, the control method of the present disclosure significantly increases the mortality activity against Riptortus pedestris by more than 2.5 times through co-treatment with the Beauveria bassiana ARP14 strain and azadirachtin compared to treatment with the Beauveria bassiana ARP14 strain alone (see Table 1, FIG. 1), but more importantly, the method increases the early infection rate of Beauveria bassiana, significantly shortening the mortality time to maximum (see Table 2, FIG. 2). Preferably, maximum mycosis may be achieved within 10 days of co-treatment with the Beauveria bassiana ARP14 strain and azadirachtin.

In the present disclosure, “co-treatment” is defined to include a single treatment with a mixture of the Beauveria bassiana ARP14 strain and azadirachtin, as well as a sequential treatment with Beauveria bassiana ARP14 strain and azadirachtin in any order, wherein the interval between treatments, if sequential, is at most 24 hours.

The treatment preferably includes, but is not limited to, a spraying treatment.

The Beauveria bassiana ARP14 strain is required to contain from 1×10⁷ to 1×10⁹ spores per unit dose (mL) in a preferred embodiment and is particularly preferably required to contain about 1×10⁸ spores per unit dose (mL). However, the treatment concentration is not limited to the above and may vary depending on circumstances and conditions such as an incidence of Riptortus pedestris, a surrounding environment, a type and growth stage of the target crops, and the like, and those skilled in the art may treat Riptortus pedestris with the Beauveria bassiana ARP14 strain at a concentration outside the range if necessary.

Azadirachtin is diluted to concentration of 0.03 g/L to 0.2 g/L for used in the treatment in a preferred embodiment. However, the treatment concentration is not limited to the above and may vary depending on circumstances and conditions such as an incidence of Riptortus pedestris, a surrounding environment, a type and growth stage of the target crops, and the like, and those skilled in the art may treat Riptortus pedestris with azadirachtin at a concentration outside the range if necessary.

Preferably, a 0.005% to 0.05% Triton X-100 solution may be used as a dilution solvent for the Beauveria bassiana ARP14 strain and azadirachtin. However, the solvent is not limited to this, and a variety of solvents known in the art as diluents for Beauveria bassiana strains or azadirachtin may be used.

The composition for the control against Riptortus pedestris includes the Beauveria bassiana strain ARP14 (KCTC 12979BP) and azadirachtin as active ingredients to increase the rate of an early fungal infection to Riptortus pedestris.

The composition may be provided in the form of a mixture of the Beauveria bassiana ARP14 strain and azadirachtin, or be provided in the form of separate packages for the Beauveria bassiana ARP14 strain and azadirachtin, respectively.

Preferably, in the composition, the Beauveria bassiana ARP14 strain is contained in an amount of 1×10⁷ to 1×10⁹ spores in a unit dose (mL), and azadirachtin is diluted at a concentration of 0.03 to 0.2 g/L when used for the treatment.

The details of each of the other elements are the same as described in the description of the control method.

The present disclosure will now be described in more detail with reference to the following examples and experimental examples. The examples and experimental examples are illustrative of the disclosure and are not intended to limit the scope of the disclosure.

### Example 1

Preparation of Beauveria bassiana ARP14 strain and azadirachtin

(1) Preparation of Beauveria bassiana ARP14 Strain

The Beauveria bassiana ARP14 strain (KCTC 12979BP) was suspended in a 0.01% Triton X-100 solution, and the spore concentration was determined using a haemocytometer (Neubauer-improved haemocytometer, Lauda-Konigshofen, Germany) to prepare a suspension having a concentration of approximately 1×10⁸ spores per mL.

### (2) Preparation of Azadirachtin

Azatrol™ (Azatrol EC, PBI-GordonCorporation) containing 1.2% of azadirachtin was diluted to a concentration of 15 ml/L using a 0.01% Triton X-100 solution as a diluent.

### Experimental Example 1

### Comparison of Mortality Effects of Co-Treatments Versus Single Treatments

The mortality effects against Riptortus pedestris by co-treatment with the Beauveria bassiana ARP14 strain and azadirachtin versus a treatment with either one of the Beauveria bassiana ARP14 strain and azadirachtin were compared. The Beauveria bassiana ARP14 strain and azadirachtin prepared in Example 1 were used. The mortality effects against 30 second-instar Riptortus pedestris were tested for each treatment.

The second-instar Riptortus pedestris were co-treated with the Beauveria bassiana ARP14 strain and azadirachtin or independently treated with only one of the Beauveria bassiana ARP14 strain and azadirachtin. The treatment method was to drop 2 μL of the solution prepared as above onto the insect's external body (topical application). Dead individuals were then visually checked and recorded daily for up to 25 days.

The results are shown in Table 1 below and FIG. 1. The co-treatment with the Beauveria bassiana ARP14 strain and azadirachtin resulted in statistically significant increases in a mortality rate for each of the second- and third-instars, as well as in a mortality rate for the total case, compared to either each treatment with one of the components alone.

TABLE 1

| | Mortality rate | | |
|---|---|---|---|
| Treatments | Second instar | Third instar | Total |
| Azatrol + B. bassiana | 0.8 (24/30) a | 1.0 (6/6) a | 1.0 (30/30) a |
| ARP14 | | | |
| Azatrol | 0.5 (16/30) a | 0.6 (9/14) ab | 0.8 (25/30) b |
| Beauveria bassiana | 0.0 (0/30) b | 0.4 (12/30) b | 0.4 (12/30) c |
| ARP14 | | | |
| Control | 0.0 (0/30) b | 0.0 (0/30) c | 0.0 (0/30) d |

### Experimental Example 2

Comparison of Changes in Infection Rate by Co-Treatment with Two Components and Independent Treatment with Only One Component

Changes in infection rates of Riptortus pedestris by co-treatment with the Beauveria bassiana ARP14 strain and azadirachtin versus each independent treatment with only one of the Beauveria bassiana ARP14 strain and azadirachtin were compared. The Beauveria bassiana ARP14 strain and azadirachtin were used as prepared in Example 1. Infection rates were verified on 30 second-instar Riptortus pedestris for each treatment.

Second-instar Riptortus pedestris were co-treated with both the Beauveria bassiana ARP14 strain and azadirachtin were treated or independently treated with one of the Beauveria bassiana ARP14 strain and azadirachtin. The treatment method was to drop 2 μL of the solution prepared as above onto the insect's external body (topical application). Fungal infections were then visually inspected and recorded daily for up to 25 days.

The results are shown in Table 2 below and FIG. 2. Infection rates were found to be higher at both days 5 and 10 when treated with azadirachtin in combination with the Beauveria bassiana ARP14 strain than when treated with the Beauveria bassiana ARP14 strain alone. Especially, the highest infection rate was found at day 10 when treated with azadirachtin in combination with the Beauveria bassiana ARP14 strain. At days 15, 20, and 25, there was no statistical difference in infection rate between the co-treatment and the independent treatment with the Beauveria bassiana ARP14 strain alone. This led to a determination that co-treatment with azadirachtin significantly sped up the infection of Riptortus pedestris with the Beauveria bassiana ARP14 strain. Thus, the increased mortality activity achieved by co-treatment of the Beauveria bassiana ARP14 strain and azadirachtin was determined to be a result of the increased infectivity of the Beauveria bassiana ARP14 strain to insects, particularly a result of the increased early infection rate.

TABLE 2

| | Mycosis rate (d) | | | | |
|---|---|---|---|---|---|
| Treatments | 5 | 10 | 15 | 20 | 25 |
| Co-treatment | 0.2 | 0.7 | 0.7 | 0.7 | 0.7 |
| | (7/30) a | (21/30) a | (21/30) a | (22/30) a | (22/30) a |
| B. bassiana | 0.0 | 0.2 | 0.4 | 0.5 | 0.5 |
| ARP14 | (0/30) b | (6/30) b | 12/30) a | (15/30) a | (15/30) a |
| Azatrol | 0.0 | 0.0 | 0.0 | 0.0 | 0.0 |
| | (0/30) b | (0/30) c | (0/30) b | (0/30) b | (0/30) b |
| Control | 0.0 | 0.0 | 0.0 | 0.0 | 0.0 |
| | (0/30) b | (0/30) c | (0/30) b | (0/30) b | (0/30) b |

The control method and composition of the present disclosure may be widely used to safely and effectively control Riptortus pedestris because with the use of only the Beauveria bassiana ARP14 strain, which is an entomopathogenic fungus that is safe for humans and the environment and selectively exhibits the activity to only the target pests, and azadirachtin, which is an eco-friendly natural insecticide, the method and composition of the present disclosure exhibit an early high infection rate and mortality effects against Riptortus pedestris.

The method of controlling Riptortus pedestris by enhancing the bioactivity of the Beauveria bassiana ARP14 strain and the composition for such control in the above-described disclosure are illustrative. The ordinarily skilled in the art to which this disclosure pertains will appreciate that diverse modifications and equivalents to the embodiments described above are possible. Therefore, it will be well understood that the present disclosure is not limited to the above-described forms, and the scope of technical protection of the present disclosure is determined by the technical ideas of the appended claims. The ordinarily skilled in the art would understand that the present disclosure covers all modifications, equivalents, and alternatives falling within the spirit of the present disclosure as defined by the claims. In addition, the terms and words used in the specification and claims should not be construed only in a conventional or dictionary sense but should be construed in a sense and concept consistent with the technical idea of the present disclosure, on the basis that the inventor can properly define the concept of a term to describe its disclosure in the best way possible.

What is claimed is:

1. A method of controlling Riptortus pedestris, the method comprising: co-treating Riptortus pedestris with Beauveria bassiana ARP14 strain (KCTC 12979BP) and azadirachtin.

2. The method of claim 1, wherein the co-treating increases an early infectivity of the Beauveria bassiana ARP14 strain to Riptortus pedestris.

3. The method of claim 1, wherein the Beauveria bassiana ARP14 strain is contained in an amount of 1×10⁷ to 1×10⁹ spores per unit dose (mL).

4. The method of claim 1, wherein azadirachtin is diluted to a concentration of 0.03 g/L to 0.2 g/L.

5. The method of claim 4, wherein a 0.005% to 0.05% Triton X-100 solution is used as a solvent for the Beauveria bassiana ARP14 strain and azadirachtin.

6. The method of claim 3, wherein azadirachtin is diluted to a concentration of 0.03 g/L to 0.2 g/L.

7. The method of claim 6, wherein a 0.005% to 0.05% Triton X-100 solution is used as a solvent for the Beauveria bassiana ARP14 strain and azadirachtin.
